(19) United States
(12) Patent Application Publication
(10) Pub. No.: US 2002/0132003 A1
(43) Pub. Date: Sep. 19, 2002

(54) Method of introducing a central nervous system stimulant to aid in the human waking process

(75) Inventors: Darcy S. O'Neil, Sarnia (CA); Theodore J. O'Neil, Sarnia (CA)

Correspondence Address: Darcy S. O'Neil, 143 Hanna Cresent, Sarnia, ON, N7S 4H4, CA

(73) Assignee: 2000801 Ontario Inc., Sarnia (CA)

(21) Appl. No.: 10/006,697

(22) Filed: Dec. 10, 2001

(30) Foreign Application Priority Data
Dec. 11, 2000 (CA) 2,327578

(51) Int. Cl. A61K 31/522; A61K 9/24

(52) U.S. Cl. 424/471; 514/263.32; 514/263.31

(57) ABSTRACT

This invention details a method for introducing a central nervous system (CNS) stimulant into the human circulatory system to aid in the waking process of the human sleep cycle. An orally administrable pharmaceutical containing an outer layer of delayed release coating encapsulates an inner core of active substance. The outer layer is designed to maintain its integrity for a period of 4 to 10 hours. For an eight hour sleep period the unit is taken 8 hours prior to the time the individual wishes to wake up. During the sleeping period the outer layer degrades in the digestive system and exposes the active substance. The active substance is then absorbed, in a single dose, and carried into the circulatory system. The active substance provides a stimulating effect to the central nervous system which provides beneficial effects in the waking process.

### FIELD OF THE INVENTION

The present invention belongs to the fields of pharmacology, medicine and medicinal chemistry, and provides a method for introducing a central nervous system (CNS) stimulant into the human circulatory system to aid in the waking process of the human sleep cycle.

### BACKGROUND OF THE INVENTION

The human sleep cycle is dictated by our circadian rhythms which have been determined by evolution. Humans are by nature are diurnal (day orientated) as opposed to nocturnal (night orientated) beings, meaning that our physiological functions are geared towards day time activity and night time rest. Normally our circadian rhythms are synchronized to one another by the internal biological clock, and entrained (daily reset) to the 24 hour day/night cycle by external time cues, namely the variation in sunlight and the increase in environmental and family activity around us. These environmental cues dictate how our internal body chemistry functions by releasing hormones to initiate sleep and waking cycles. It is therefore an unnatural occurrence for humans to wake prior to daylight. At this time the core temperature of the body is at it's lowest and the desire to sleep is at its strongest.

However, as modern society changes the requirement for many individuals to function outside of these normal rhythms is increasing. The accepted North American work schedule is from 8:00 AM to 5:00 PM so the average waking time for most people is from 6:00 AM to 8:00 AM, which falls within the range of the circadian rhythm where we have the strongest desire to sleep.

The Effects of Xanthines on Human Physiology

The process of sleep is still a major scientific mystery however in recent years many discoveries have been made regarding the physiology of sleep. It is know that adenosine is created in the brain, it binds to adenosine receptors. The binding of adenosine causes drowsiness by slowing down nerve cell activity. In the brain, adenosine binding also causes blood vessels to dilate (to let more oxygen in during sleep).

To a nerve cell, caffeine looks like adenosine. Caffeine therefore binds to the adenosine receptor. However, it doesn't slow down the cell's activity like adenosine would. So the cell cannot “see” adenosine anymore because caffeine is taking up all the receptors adenosine binds to. So instead of slowing down because of the adenosine level, the cells speed up. You can see that caffeine also causes the brain's blood vessels to constrict, because it blocks adenosine's ability to open them up.

With caffeine blocking the adenosine receptors you now have increased neuron firing in the brain. The pituitary gland sees all of the activity and thinks some sort of emergency must be occurring, so it releases hormones that tell the adrenal glands to produce adrenaline (epinephrine). Adrenaline has a variety of effects on human biology including:

Your pupils dilate

Your breathing tubes open up

Your heart beats faster

Blood vessels on the surface constrict

Blood pressure rises.

Blood flow to the stomach slows

The liver releases sugar into the bloodstream for extra energy

Caffeine also increases dopamine levels in the same way that amphetamines do. (e.g. heroine and cocaine) Dopamine is a neurotransmitter that, in certain parts of the brain, activates the pleasure centre leaving you with a sense of well being.

The function of adrenaline is to prepare the body for a physical response commonly called the flight or fight response. This stimulated response can be an important aid in the waking process.

During our sleep cycles the brain shuts down many physiological functions to provide a state of recuperation for the body. If were are forced to wake earlier than our circadian rhythms dictate we find it very difficult because our body is still in a state of rest and many of the function necessary for activity have not been initiated. By introducing a CNS stimulant, such as caffeine, just prior to the time we are required to awake we can reduce many of the symptoms associated with waking at unnatural times by stimulating functions required for activity.

Improvement on Previous Designs

To date Xanthine and derivative CNS pharmaceuticals have been designed to provide either an immediate release of the stimulant into the body or provide an initial dose of stimulant and then provide a sustained release of the active component over a period of time. These pharmaceuticals are generally taken during the active cycles of the human circadian rhythms to improve alertness and reduce the urge to sleep or rest.

This invention is designed to alleviate the issues associated with waking up, including inability to concentrate, moodiness and irritability, confusion, reduced body temperature, slower reaction times, and lower mental capacity by providing a dose of caffeine or other stimulant, prior to awakening, to prepare the body for activity. The major design improvement is that this product is used as an awakening agent instead of a sustaining agent.

Detailed Description of Operation

The invention is designed to be taken orally in the form of a tablet or a capsule containing a multitude of micro tablets. The outer layer of the invention is a biodegradable coating, designed from a variety of natural and synthetic compounds, with properties that allow it to decompose at a specified rate in the human digestive system leading to release time of the core stimulant within 4 to 10 hours. This “decomposition” can be performed by a variety of chemical or physical actions including erodible designs, chemical decomposition or pH dependant release.

The release of the core stimulant from the invention will be of the burst or chrono-release type. Basically, the core of stimulant will be release into the system in a short period of time, preferably for the most effective operation 80% to 90% of the drug should be released in a 10 to 30 minute period. However, a slower release profile still results in a waking experience but without some beneficial effects.

For proper operation of the invention, the user will be required to determine the time they wish for the caffeine to begin its effects. For example, if the required time for the individual to wake up on Monday morning is 6:00 AM then the invention should be taken at 10:00 PM Sunday night, assuming an 8 hour release is used.

Detailed Description of Specific Embodiments

To form a tablet in accordance with the invention, the stimulant will be mixed with tableting excipients e.g. one or more of the standard excipients such as diluents, fillers, lubricants, binding agents, flow aids, disintegrating agents, surface active agents or water soluble polymeric materials and any other materials used to produce a tablet by those skilled in the art.

To produce tablets in accordance with the invention, excipient(s) may be mixed or blended, using conventional procedures, e.g. using a bin-blender and the resulting mixture compressed according to conventional tableting procedure using a suitable size tableting mould. Tablets can be produced using conventional tableting machines, such as a standard single punch machine or rotary tablet machine.

The time delayed coating consists of a biodegradable material or combination of materials. Two such materials used in combination are PVAP (polyvinyl acetate phthalate) and Ethylcellulose.

Ethylcellulose is a hydrophobic coating material that can be used to achieve a variety of coating applications including taste masking, moisture barrier, and controlled release of multi-particulate dosage forms. PVAP is a pH dependant polymer that dissolves in gastric Fluids with a pH of 6.8 or above.

### EXAMPLE 1

Caffeine (100 mg), 86.4 mg of microcrystalline cellulose, 50.4 mg of Starch 1500, and 1.2 mg, 1.2 mg of carbosil and 1.2 mg of Sodium Benzoate are mixed thoroughly. Stearic acid (1.2 mg) is added and thoroughly mixed for another 5 min. The granular mixture is formed into tablet cores of 6.8 mm diameter, weighing 240 mg each using a Manesty F-1 rotary tablet press. The cores show a disintegration time lower than 5 min. in water, a hardness of 18 KP, 6KNCF and 0.2KNEF.

The cores are heated to 50 C. and then cooled and maintained at 40C. The coating layer is applied onto the cores in two steps, using an O'Hara Labcoat II automatic coating pan. In the first step, the cores are coated with an aqueous solution of 15% PVAP at a rate of 30 grams per minute for a period of 60 minutes. This results in a 10% coating of PVAP on the tablet cores.

In the second step, the cores are treated with an aqueous solution of 15% Ethylcellulose at a rate of 30 grams per minute for a period of 70 minutes resulting in a 12.5% Ethylcellulose coating.

The formulation for the tablet core is:

| | |
|---|---|
| Caffeine | 41.49% |
| Microcrystalline Cellulose 90 μm | 36.01% |
| Starch 1500 | 21.00% |
| Stearic Acid | 0.50% |
| Cabosil | 0.50% |
| Sodium Benzoate | 0.50% |

Formulation for 8 Hour Time Delayed Coating:

| | |
|---|---|
| PVAP | 10% by weight coating |
| Ethylcellulose | 12.5% by weight coating |

### EXAMPLE 1

(10% PVAP & 12.5% Ethylcellulose Coating):

This test was conducted using subjects with varying levels of daily caffeine consumption, usually in the form of coffee. The tablets were coated with 10% PVAP & 12.5% Ethylcellulose coating. The pills were taken at 9:00 PM each night to ensure that the waking time would be in a the core sleeping time. In this manner the effects of the caffeine could be observed more clearly since the subjects involved in the test rarely wake up at this time and data points would not be confused with regular waking times. The total number of hours of sleep time are listed below:

| | | User #1 | | User #2 | | User #3 | |
|---|---|---|---|---|---|---|---|
| \|Day 1 | \| | 7 Hrs 35 min | \| | 7 Hrs 50 min | \| | 8 Hrs 10 min | \| |
| \|Day 2 | \| | 7 Hrs 50 min | \| | 7 Hrs 55 min | \| | 8 Hrs 05 min | \| |
| \|Day 3 | \| | 7 Hrs 45 min | \| | 7 Hrs 50 min | \| | 8 Hrs 05 min | \| |

User #4 - 7 Hours 50 minutes (Coffee Drinker 400 mg/day)
User #5 - 8 Hours 05 minutes (Coffee Drinker +500 mg/day)
User #6 - No waking point (Heavy coffee drinker 900 mg/day)

User #1 and #2 were female consuming approximately 200 mg of caffeine per day with a normal waking time of 7:00 AM and an average body weight of 135 lbs.

User #3 was a male who averages 500 mg of caffeine per day with a normal waking time of 7:00 AM and an average body weight of 160 lbs.

All results are rounded to the nearest 5 minute interval.

### EXAMPLE 2

(10% PVAP and 10% Ethylcellulose Coating):

A second test was conducted to demonstrate that by varying the Ethylcellulose coating we could modify the release time of the caffeine and thus the waking effect. In this example tablets were taken at 9:00 PM and resulted in an early morning waking time approximately 6 hours after ingestion.

| | | User #1 | | User #2 | | User #3 | |
|---|---|---|---|---|---|---|---|
| \|Day 1 | \| | 6 Hrs 15 min | \| | 5 Hrs 50 min | \| | 6 Hrs 15 min | \| |
| \|Day 2 | \| | 6 Hrs 05 min | \| | 5 Hrs 55 min | \| | 6 Hrs 30 min | \| |
| \|Day 3 | \| | 6 Hrs 00 min | \| | 5 Hrs 25 min | \| | 6 Hrs 30 min | \| |

User #1 and #2 were female, consuming approximately 200 mg of caffeine per day with a normal waking time of 7:00 AM and an average body weight of 135 lbs.

User #3 was a male who averages 500 mg of caffeine per day with a normal waking time of 7:00 AM and an average body weight of 160 lbs.

All results are rounded to the nearest 5 minute interval.

The embodiments of the invention in which an exclusive property or privilege is claimed are defined as follows:

1. A method to deliver an orally administrable pharmaceutical, containing an outer layer of time delayed coating and a core of active substance to assist in the waking process of the human sleep cycle.

2. An outer layer according to claim 1 that delays the release of the active substance for a period of 4 to 10 hours after administering.

3. A dosage form according to claim 1 in which the active substance is a single unit, such as a pill, or a capsule containing a quantity of micro encapsulated units.

4. The active substance, according to claim 1, being a central nervous system stimulant, from the Xanthine family of compounds, specifically: Caffeine (1,3,7-trimethylxanthine), Theobromine (3,7-dimethylxanthine), Guaranine (tetra-methylxanthine), Paraxanthine and Mateine.

5. The active substance, according to claim 1, being a central nervous system stimulant from the following list Methylphenidate (Ritalin), Ephedrine (alpha-[1-(Methylamino)ethyl]benzene-methanol) and Pseudoephedrine.

6. The active substance from claim 1 is a mixture of central nervous system stimulants from claim 4 and 5.

7. The active substance from claim 1 is in a dosage range of 5 mg to 500 mg per unit.

8. The outer layer according to claim 2 wherein the coating substance is selected from natural and synthetic biologically degradable materials that cause a delay in the release of the active substance.

9. The outer layer according to claim 8 wherein the coating is a combination of natural and synthetic materials that cause a delay in the release of the active substance.

10. A pharmaceutical composition of claim 1 further comprising, in place of a corresponding amount of active ingredient 1 to 95 wt % of an inert bulking excipient, pharmaceutically acceptable in oral compositions.
